(19) United States
(12) Patent Application Publication
(10) Pub. No.: US 2025/0014704 A1
(43) Pub. Date: Jan. 9, 2025

(54) METHOD AND SYSTEM FOR PROVIDING PRESCRIPTION DRUG PREPARATION, DISTRIBUTION AND DELIVERY SERVICES THROUGH ELECTRONIC PRESCRIPTION

(71) Applicant: LEMONHEALTHCARE LTD, Seoul (KR)

(72) Inventor: Byung Jin HONG, Seoul (KR)

(21) Appl. No.: 18/896,292

(22) Filed: Sep. 25, 2024

Related U.S. Application Data
(63) Continuation of application No. 17/703,061, filed on Mar. 24, 2022.

(30) Foreign Application Priority Data
Oct. 29, 2021 (KR) 10-2021-0146252

(51) Int. Cl. G16H 20/10; G06Q 20/10; G06Q 20/38; G06Q 20/40; G16H 40/20; G16H 40/67

(52) U.S. Cl. CPC G16H 20/10; G06Q 20/102; G06Q 20/382; G06Q 20/4014; G16H 40/20; G16H 40/67

(57) ABSTRACT

A method of providing prescription drug preparation, distribution and delivery services through an electronic prescription. The method includes: executing, by a user terminal, a patient-only application to select a hospital where to receive medical care and whether to receive remote medical care, and transmitting a result of the selection to a cloud server; determining, by the cloud server, whether a hospital appointment and remote medical care are possible based on a selection result received from the user terminal, and requesting, by the cloud server, an appointment and remote medical care to a hospital server available of provision of medical care based on a determination result; proceeding with, by the hospital server, the appointment of the remote medical care and the remote medical care, requesting, by the hospital server, payment of medical expenses from the cloud server, and requesting, by the cloud server, confirmation of medical expenses from the user terminal.

### CROSS-REFERENCE TO RELATED APPLICATIONS

This application is a continuation in part application of U.S. Non-Provisional patent application Ser. No. 17/703,061, filed on Mar. 24, 2024, which claims priority from Korean Patent Application No. 10-2021-0146252, filed Oct. 29, 2021, the entire contents of which are hereby incorporated by reference.

### TECHNICAL FIELD

The present disclosure relates to a method and a system for providing prescription drug preparation and distribution through electronic prescription and for providing a prescription drug delivery service through authentication, and more particularly, to a method and a system for providing prescription drug preparation, distribution and delivery services through electronic prescription that is provided with an authentication procedure and a medication guide procedures to safely prepare, distribute, and deliver a drug without a need to visit a pharmacy after medical care.

### BACKGROUND

Remote medical care does not guarantee a quality of medical care and may cause a problem about unclear legal responsibility for a result of the medical care. Due to such drawbacks, the introduction of the remote medical care has been withheld despite various advantages. However, due to the recent pandemic, the need for the remote medical care is emerging, and governments are actually making progress to allow remote medical care. In the meantime, the demand for services in which medicines are delivered without visiting a pharmacy after remote medical care is expected to increase. In addition, there will be an increasing need for a service of delivering a drug without visiting a pharmacy after remote medical care.

Also, the wait for receiving a prescription drug after receiving a medication guide at the door-to-door pharmacy is not long in the case of small hospitals and clinics, but the wait is long in the case of secondary and tertiary hospitals due to a large number of patients. In addition, the patients of the secondary and tertiary hospitals need to receive the drug, return to the hospitals, and go back home by car, which is cumbersome. There is need to address this inconvenience.

### SUMMARY

In view of the above, the present disclosure provides a method and a system for providing prescription drug preparation and distribution through electronic prescription and for providing a prescription drug delivery service through authentication, so that a patient can receive a prescription drug anytime anywhere after medical care.

According to embodiments of the present disclosure, there is provided a method of providing prescription drug preparation, distribution and delivery services through an electronic prescription. The method includes: executing, by a user terminal, a patient-only application to select a hospital where to receive medical care and whether to receive remote medical care, and to transmit a result of the selection to a cloud server; determining, by the cloud server, whether a hospital appointment and remote medical care are possible based on a selection result received from the user terminal, and requesting, by the cloud server, an appointment and remote medical care to a hospital server available of provision of medical care based on a determination result; proceeding with, by the hospital server, the appointment of the remote medical care and the remote medical care, requesting, by the hospital server, payment of medical expenses from the cloud server, and requesting, by the cloud server, confirmation of medical expenses from the user terminal; making, by the user terminal, payment for medical care and prescription inquiry, requesting, by the user terminal, drug dispensing from the cloud server, and requesting and receiving, by the cloud server, an electronic prescription from the cloud server; providing, by the cloud server, a pharmacy list to the user terminal, selecting, by the user terminal, information on a pharmacy and a delivery method, transmitting, by the user terminal, a selection result to the cloud server, and transmitting, by the cloud server, the electronic prescription to a pharmacy server; and checking, by the pharmacy server, stock based on the received electronic prescription, wherein when a drug included in the electronic prescription is in stock, the pharmacy server transmits a drug QR code to a drug distribution server retrieves the corresponding drug by checking the drug QR code and transmits the drug to the pharmacy server, and when a drug included in the electronic prescription in stock, the pharmacy server payment of a drug cost from the user terminal.

According to the present disclosure, since a patient selects and goes to a pharmacy to receive a prescription drug through a user terminal, the waiting time to receive the prescription medicine may be almost zero, and there is no need to keep paper prescriptions. Furthermore, the patient is able to inquire about a prescription and pay a drug cost and a delivery cost through the user terminal, so it is possible to receive the drug in a non-face-to-face process.

In addition, the patient is able to ask for drug prescription after prepayment of the drug cost and to receive a delivered drug and a medication guide at a desired time without need to visit the pharmacy to receive the drug.

In addition, since drug inventory information is shared between a pharmacy server and a drug distribution server, pharmacies capable of receiving electronic prescriptions may provide a prescription drug and manage inventory in real time.

In addition, it is possible to prevent erroneous delivery of a drug through an electronic prescription-based authentication procedure, and allow medication guide through an online medication guide procedure.

Since patients do not need to visit hospitals or pharmacies in person, it is effective in preventing infectious diseases by minimizing contact as well as saving time and money.

### BRIEF DESCRIPTION OF THE DRAWINGS

FIGS. 1 and 2 are flowcharts illustrating a method of providing prescription drug preparation, distribution and delivery services through an electronic prescription according to an embodiment of the present disclosure.

FIG. 3 is a configuration diagram illustrating a system for providing services for preparing, distributing and delivering prescription drugs through an electronic prescription according to an embodiment of the present disclosure.

FIG. 4 is a conceptual diagram illustrating a method of providing prescription drug preparation, distribution and delivery services through an electronic prescription according to an embodiment of the present disclosure.

FIG. 5 is a flowchart illustrating a method of providing prescription drug preparation, distribution and delivery services through an electronic prescription according to another embodiment of the present disclosure.

FIG. 6 is a block diagram illustrating a system for providing services for preparing, distributing and delivering a prescription drug through an electronic prescription according to another embodiment of the present disclosure.

FIG. 7 is a flowchart illustrating an authentication process according to another embodiment of the present disclosure.

FIG. 8 is a flowchart illustrating an authentication process according to another embodiment of the present disclosure.

FIG. 9 is a flowchart illustrating an authentication process according to another embodiment of the present disclosure.

### DETAILED DESCRIPTION OF EXEMPLARY EMBODIMENTS

While a specific structural or functional description with respect to embodiments according to the present disclosure disclosed in this specification is merely provided for the purpose of describing the embodiments of the present disclosure, there are various modifications capable of replacing the embodiments, and the present disclosure is not limited to the embodiments described in this specification.

While the embodiments according to the present disclosure are susceptible to various modifications and alternative forms, specific embodiments thereof are shown by way of examples in the drawings and will herein be described in detail. It should be understood, however, that there is no intent to limit the present disclosure to the particular forms disclosed, but on the contrary, the present disclosure is to cover all modifications, equivalents, and alternatives falling within the spirit and scope of the invention.

The terminology used herein is for the purpose of describing particular embodiments only and is not intended to be limiting of the disclosure. The singular forms include plural referents unless the context clearly dictates otherwise. In this specification, terms such as “comprise” or “have” are intended to designate that a feature, number, step, operation, component, part, or combination thereof described herein exists, but it should be understood that they do not preclude the possibility of the presence or addition of numbers, steps, operations, components, parts, or combinations thereof.

Hereinafter, embodiments of the present disclosure will be described in detail with reference to the accompanying drawings.

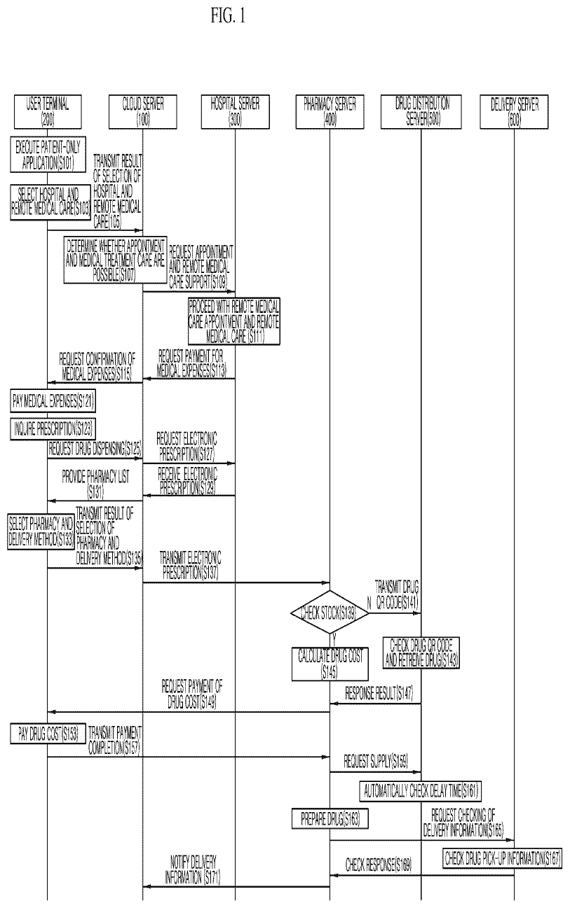

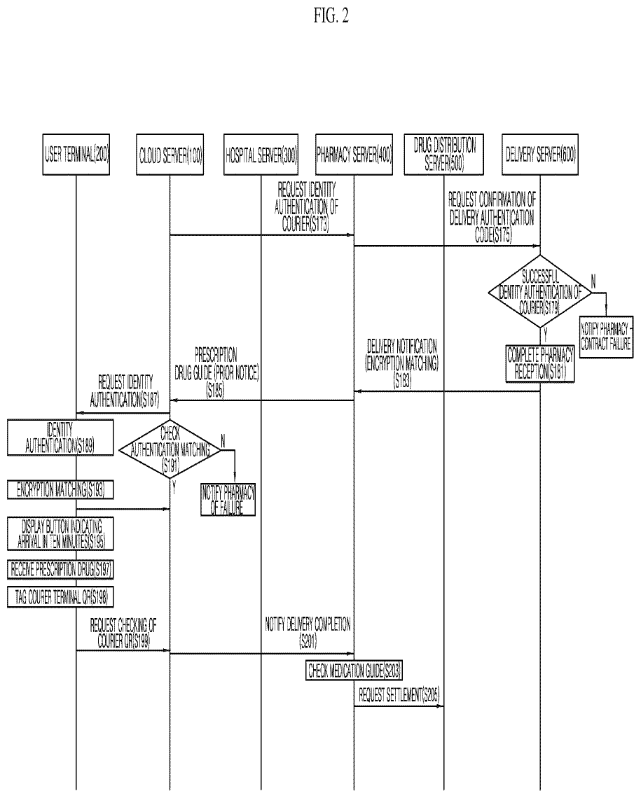

FIGS. 1 and 2 are flowcharts illustrating a method of providing prescription drug preparation, distribution and delivery services through an electronic prescription according to an embodiment of the present disclosure.

Referring to FIGS. 1 and 2, a user terminal 200 executes a patient-only application in operation S101, selects a hospital where to receive medical care from the patient-only application and whether to receive a remote medical care in operation S103, and transmits a selection result to a cloud server in operation S105. The drug delivery service in the present disclosure is not limited to remote medical care, and the drug delivery service may also be a drug delivery service after general medical care. When drug delivery starts, the patient-only application may provide a drug delivery status including a delivery location, a reception status, and order information.

The cloud server 100 checks whether a hospital appointment and remote medical care are possible according to the selection result received from the user terminal 200 in operation S107, and requests, based on a confirmation result, the appointment and the remote medical care from the hospital server 300 of a hospital available to proceed with the remote medical care in operation S109.

The hospital server 300 proceeds with the remote medical care appointment and the remote medical care in operation S111, and requests payment of medical expenses from the cloud server 100 in operation S113. The cloud server 100 requests confirmation of the medical expenses from the user terminal 200 in operation S115, and a user terminal makes the payment of the medical expenses in operation S121 and inquires prescription in operation S123, requests drug dispensing from the cloud server 100 in operation S125, and the cloud server 100 requests an electronic prescription from the hospital server 300 in operation S127 and receives the electronic prescription in operation S129.

The cloud server 100 provides the user terminal 200 with a list of pharmacies capable of delivering drug in operation S131, and the user terminal 200 selects a pharmacy and a delivery method in operation S133 and transmits a selection result to the cloud server in operation S135, and the cloud server 100 transmits an electronic prescription to a pharmacy server 400 in operation S137.

The pharmacy server 400 checks a stock based on the received electronic prescription in operation S139. When a drug included in the electronic prescription is not in stock, the pharmacy server transmits a drug QR code to the drug distribution server 500 in operation S141 and the drug distribution server retrieves the drug by checking the drug QR code and transmits the drug to the pharmacy server 400 in operation S143. When a drug included in the electronic prescription is in stock, the pharmacy server 400 calculates a drug cost in operation S145 and requests payment of the drug cost from the user terminal 200 in operation S149.

When the user terminal 200 proceeds with drug cost payment in operation S153, payment completion is transmitted to the pharmacy server 400 in operation S157, the pharmacy server 400 requests a supply from the drug distribution server 500 in operation S159, and the drug distribution server 500 automatically checks a delay time in operation S161.

The pharmacy server 400 prepares a drug, requests checking of delivery information from the delivery server 600, the delivery server 600 checks drug pickup information and requests confirmation from the pharmacy server 400, and the pharmacy server 400 transmits delivery information to the cloud server 100 in response.

Referring to FIG. 2, the cloud server 100 requests identity authentication of a courier from the pharmacy server 400 in operation S173, and the pharmacy server 400 requests confirmation of a delivery authentication code from the delivery server in operation S175. When the identity authentication of the courier succeeds in operation S179, the delivery server completes pharmacy reception in operation S181 and transmits a delivery notification to the pharmacy server (in an encryption matching manner) in operation S183.

The pharmacy server 400 transmits a prescription drug guide (prior notice) to the cloud server in operation S185, the cloud server requests identity authentication from the user terminal in operation S187, and the user terminal performs identity authentication in operation 189 and encryption matching in operation S193.

The cloud server 100 checks authentication matching in operation S191. When the authentication matching fails, the cloud server 100 notifies the pharmacy of the failure, the user terminal 200 displays a button indicating arrival in ten minutes. When the prescription is received, the user terminal tags a courier terminal QR and requests checking of a courier QR from the cloud server 100 in operation S199.

When delivery completion is received from the cloud server 100, the pharmacy server 400 checks a medication guide in operation S203 and requests settlement from the delivery server 500 in operation S205.

In the present disclosure, in order to prevent erroneous delivery, a method of authenticating reception of a drug is important and the method may be implemented as any of various authentication methods. In the present disclosure, an authentication code may be activated/deactivated according to a status to ensure security. The authentication code is deactivated (non-exposed) before a specific button or a specific step is performed. For example, the authentication code of the user terminal may be activated at a specific time (e.g., before ten minutes) based on an expected delivery time from the delivery server.

It is possible to check whether the authentication codes of the courier terminal and the user terminal match each other by tagging the courier terminal and the user terminal.

Details of a method of receiving a drug are as follows.

TABLE 1

| Division | How to authenticate drug reception | |
|---|---|---|
| Pharmacy server | Delivery server | User terminal |
| Upon completion of | (Disable) Encrypted | (Disable) Encrypted |
| drug dispensing | authentication code | authentication code |
| (completion) | Pick-up by courier | (Receiving) message to |
| | | notify delivery |
| | | authentication time |
| Delivery server | Courier terminal | User terminal (before |
| | | receiving) |
| Select order > | Encrypted authentication | Encrypted authentication |
| Contract | code-confirmation button | code (deactivated) |
| | button indicating arrival | |
| | in ten minute | |
| User terminal (after | Courier terminal | Pharmacy server |
| reception) | | |
| Activation + | Encrypted authentication | Encrypted authentication |
| Matching | code (activated) | code (completion of |
| | Discard authentication | confirmation) |
| | code after reception of | |
| | drug | |

As an additional embodiment of a drug receiving method, it is also possible to send an authentication number in a notification message to the courier terminal, and to match the input number by pairing to the user terminal. When a user who receives the authentication number or encryption key sent to the notification talk of the courier terminal clicks the deactivated button, an authorization number input window is provided, and the button is activated only after entering the authorization number of the courier terminal. A courier terminal authentication number (encryption key) and a user terminal authentication number (encryption key) are a pair. Thus, when different types of data are matched, a warning notification is output indicating an unallowed user and a message indicating that a contract cannot be made may be transmitted. In this case, the pharmacy that has dispensed the drug may cancel the prescription and induce the user to cancel the payment. The notification message may be implemented in any of notification methods such as a short message service (SMS), a long message service (LMS), a multimedia message service (MMS), Kakao Talk Notification, and a push notification

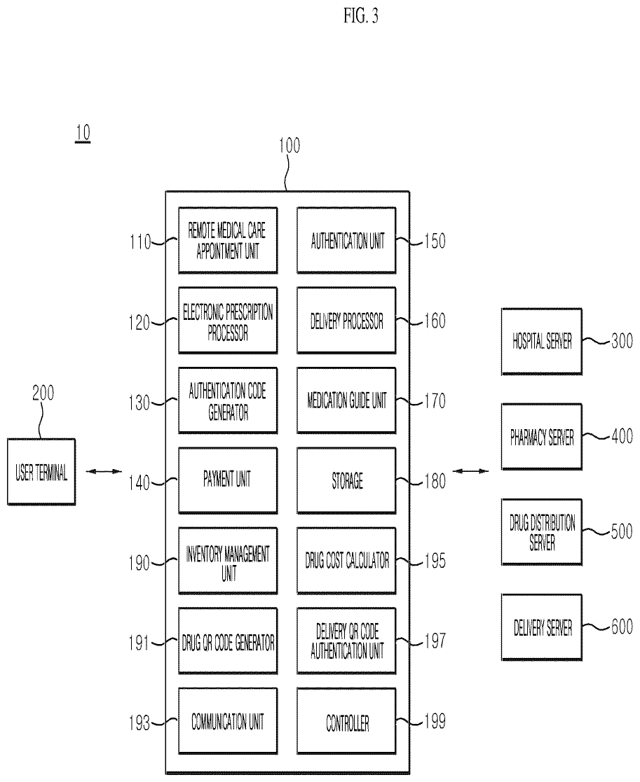

FIG. 3 is a diagram for explaining a system for providing prescription drug preparation, distribution and delivery services through an electronic prescription according to an embodiment of the present disclosure.

Referring to FIG. 3, a system for providing prescription drug dispensing, distribution and delivery service through an electronic prescription is a cloud server 100, a user terminal 200, a hospital server 300, a pharmacy server 400, a drug distribution server 500, and a delivery server 600.

The cloud server 100 includes a remote medical care appointment unit 110, an electronic prescription processor 120, an authentication code generator 130, a payment unit 140, an authentication unit 150, a delivery processor 160, a medication guide unit 170, a storage 180, an inventory management unit 190, a drug QR code generator 191, a communication unit 193, a drug cost calculator 195, a delivery QR code authentication unit 197, and a controller 199.

The remote medical care appointment unit 110 may determines whether a hospital appointment and a remote medical care are possible based on a selection result received from the user terminal 200, such as a hospital where to receive medical care and whether to receive remote medical care. Based on a determination result, the remote medical care appointment unit 110 may request an appointment and a remote medical care from a server of a hospital capable of providing medical care.

The electronic prescription processor 120 requests and receives an electronic prescription from the hospital server upon the user terminal 200 requesting drug dispensing from the cloud server 100, and provides the electronic prescription to the pharmacy server 400 upon receiving information on a pharmacy and a delivery method from the user terminal 200.

The authentication code generator 130 may generate a unique authentication code using the electronic prescription received from the hospital server. The authentication code may be generated by encrypting a unique ID of the electronic prescription. When the user terminal and the courier terminal receive the delivered drug through the generated authentication code, it may be determined whether or not the authentication code matches, thereby preventing erroneous delivery of the drug.

When a request for payment of medical expenses is received from the hospital server, the payment unit 140 may request the payment of medical expenses from the user terminal.

The authentication unit 150 may receive authentication information from the user terminal 200 to determine whether identity authentication succeeds, and when the identity authentication fails, the authentication unit 150 may notify the pharmacy server of an authentication failure.

The delivery processor 160 may request the user terminal 200 to confirm reception of delivery and transmit a result of delivery of the prescription drug to the pharmacy server. When the reception of delivery is not confirmed, the delivery processor 160 may check the courier's contact. The delivery processor 160 may provide the user terminal with a date and a time, which are selected and desired by the user terminal to receive the drug, and with drug delivery information as a notification message.

In order to provide a medication guide according to the electronic prescription, the medication guide unit 170 may provide a medication guide image to the user terminal upon receiving the medication guide image from the pharmacy server. Upon receiving confirmation of reception of the medication guide from the user terminal, the medication guide unit 170 may notify the pharmacy server 300 of medication guide completion in response.

The storage 180 may store the generated authentication code and store the received medication guide image.

When the inventory management unit 190 inputs at least one of a supply plan, inventory information, and a request quantity, the drug distribution server having received a corresponding order may automatically calculate a consumption quantity and an order quantity based on information input by a pharmacy server, which placed the order, and creates an order sheet and then a corresponding drug may be supplied to a pharmacy that placed the order. The inventory management unit 190 may exchange information with the drug distribution server in real time. When the electronic prescription is received, the inventory management unit 190 may check the inventory of the corresponding drug. When the inventory is insufficient, the inventory management unit 190 may transmit a drug list included in the electronic prescription to the drug distribution server and store information related to drugs included in the drug list. The information related to the drugs may include information on effects and efficacy of a plurality of drugs. In addition, stock quantity information on the drug may be stored, and replenishment scheduling information according to drug replenishment request information of the pharmacy server may be stored. The inventory management unit 190 may request drug replenishment from the drug distribution server when a quantity of drugs stored in a drug packet unit is less than a predetermined quantity. The drug QR code generator 191 is a QR code generated based on a drug list described in the received electronic prescription, and patient information is not included in the generated drug QR code. The drug cost calculator 195 may calculate a drug cost based on prescription information and request a calculated cost from the cloud server 100. When a delivery QR confirmation is requested from the user terminal, the delivery QR code authentication unit 197 may authenticate a delivery QR code of a courier. When the delivery QR code of the courier is authenticated, the delivery QR code authentication unit 197 may notify the pharmacy server of delivery completion. The communication unit 193 may exchange data with the user terminal, the hospital server, and the pharmacy server through a wired/wireless communication module. The controller 199 controls each configuration of the cloud server.

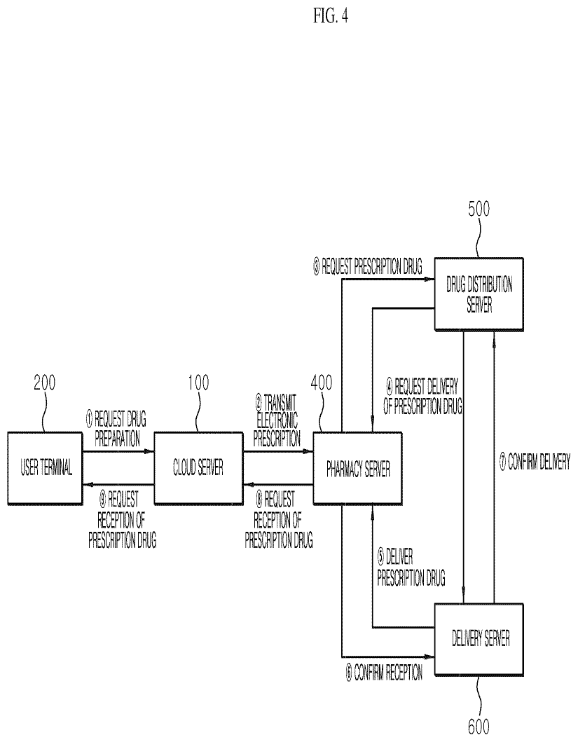

FIG. 4 is a conceptual diagram illustrating a method of providing prescription drug preparation, distribution and delivery services through an electronic prescription according to an embodiment of the present disclosure.

Referring to FIG. 4, in the method of providing prescription drug preparation, distribution and delivery services through an electronic prescription, when a user terminal 200 requests drug preparation from a cloud server 100, the cloud server 100 transmits an electronic prescription, received from a hospital server, to a pharmacy server 400. The pharmacy server 400 requests a prescription drug from a drug distribution server 500, and the drug distribution server 500 requests delivery of the drug from a delivery server 600. The delivery server 600 delivers the prescription drug to the pharmacy server 400 and confirms the delivery for the drug delivery server 500 upon receiving a reception confirmation. The pharmacy server 400 may request reception of the prescription drug through the cloud server 100, and a patient may set a visit time to a pharmacy through the user terminal 200 and receive the prescription drug at the pharmacy. The pharmacy server 400 may check whether the delivered prescription drug is in stock, and may request the corresponding prescription drug from a drug wholesale server in real time.

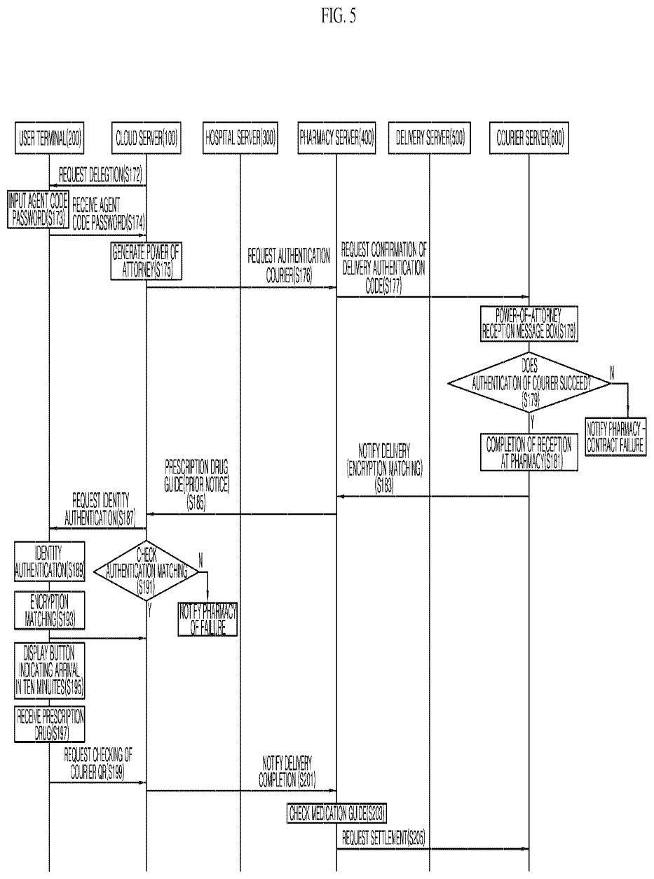

FIG. 5 is a flowchart illustrating a method of providing prescription drug preparation, distribution and delivery services through an electronic prescription according to another embodiment of the present disclosure;

FIG. 5, which is another embodiment of FIG. 2, relates to an authentication method through a power of attorney (POA).

A cloud server 100 makes a delegation request to a user terminal 200 in operation S172, and the user terminal 200 inputs an agent code password in operation S173. The user terminal 200 transmits the input agent code password to the cloud server 100, and the cloud server generates a POA based on the input agent code password in operation S175. The cloud server 100 requests identity authentication of a courier from a pharmacy server 400 in operation S176, and the pharmacy server 400 requests confirmation of a delivery authentication code from a delivery server 600 in operation S177.

The delivery server 600 receives the POA through a power-of-attorney receiving message box in operation S178, and performs authentication of the courier. When the identity authentication succeeds, the delivery server 600 completes pharmacy reception in operation S181, and when the identity authentication fails, the delivery server notifies a corresponding pharmacy of the failure. The delivery server 600 transmits a delivery notification to the pharmacy server (in an encryption matching manner) in operation S183.

The pharmacy server 400 transmits a prescription drug guide (prior notice) to the cloud server in operation S185, the cloud server requests identity authentication from the user terminal in operation S187, and the user terminal performs identity authentication in operation S189 and encryption matching in operation S193.

The cloud server 100 checks authentication matching in operation S191. When the authentication matching fails, the cloud server 100 notifies the pharmacy of the failure, the user terminal 200 displays a button indicating arrival in ten minutes. When the prescription is received, the user terminal tags a courier terminal QR and requests checking of a courier QR from the cloud server 100 in operation S199.

When delivery completion is received from the cloud server 100, the pharmacy server 400 checks a medication guide in operation S203 and requests settlement from the delivery server 500 in operation S205.

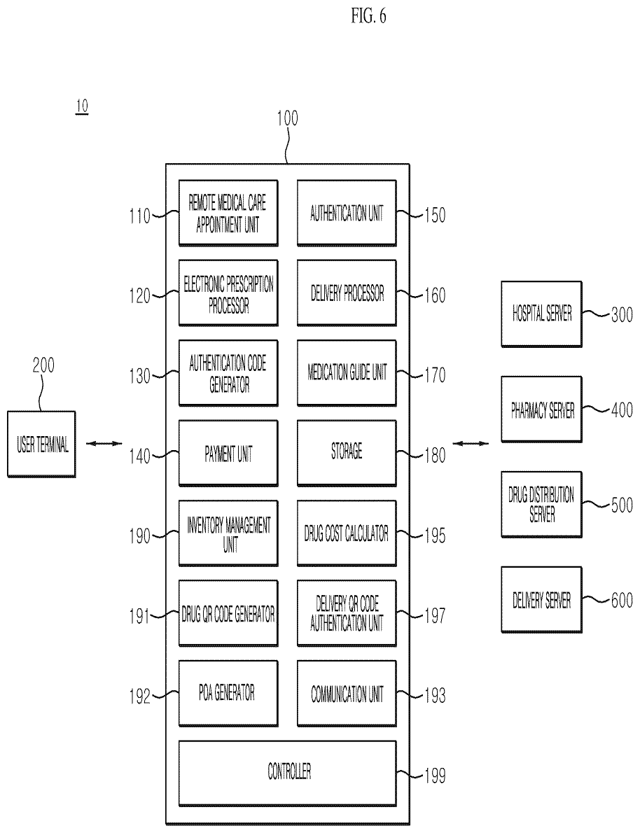

FIG. 6 is a block diagram illustrating a system for providing services for preparing, distributing and delivering a prescription drug through an electronic prescription according to another embodiment of the present disclosure.

Referring to FIG. 6, a system 10 for providing services for preparing, distributing and delivering a prescription drug through an electronic prescription includes a cloud server 100, a user terminal 200, a hospital server 300, a pharmacy server 400, a drug distribution server 500, and a delivery server 600.

The cloud server 100 includes a remote medical care appointment unit 110, an electronic prescription processor 120, an authentication code generator 130, a payment unit 140, an authentication unit 150, a delivery processor 160, a medication guide unit 170, a storage 180, an inventory management unit 190, a drug QR code generator 191, a power of attorney (POA) generator 192, a communication unit 193, a drug cost calculator 195, a delivery QR code authentication unit 197, and a controller 199.

The remote medical care appointment unit 110 may determines whether a hospital appointment and a remote medical care are possible based on a selection result received from the user terminal 200, such as a hospital where to receive medical care and whether to receive remote medical care. Based on a determination result, the remote medical care appointment unit 110 may request an appointment and a remote medical care from a server of a hospital capable of providing medical care.

when the user terminal 200 requests drug preparation from the cloud server 100, the electronic prescription processor 120 receives an electronic prescription from the hospital server. When information on a pharmacy and a delivery method are received from the user terminal 200, the electronic prescription processor 120 transmits the electronic prescription to the pharmacy server 400.

The authentication code generator 130 may generate a unique authentication code using the electronic prescription received from the hospital server. The authentication code may be generated by encrypting a unique ID of the electronic prescription. When the user terminal and the courier terminal receive the delivered drug through the generated authentication code, it may be determined whether or not the authentication code matches, thereby preventing erroneous delivery of the drug.

When a request for payment of medical expenses is received from the hospital server, the payment unit 140 may request the payment of medical expenses from the user terminal.

The authentication unit 150 may receive authentication information from the user terminal 200 to determine whether identity authentication succeeds, and when the identity authentication fails, the authentication unit 150 may notify the pharmacy server of an authentication failure.

The delivery processor 160 may check reception of delivery from the user terminal 200 and transmit a result of delivery of the prescription drug to the pharmacy server. When the reception of delivery is not confirmed, it is possible to check a corresponding courier's contact information. The delivery processor 160 may provide the user terminal with a date and a time, which are selected and desired by the user terminal to receive the drug, and with drug delivery information as a notification message.

In order to provide a medication guide according to the electronic prescription, the medication guide unit 170 may provide a medication guide image to the user terminal upon receiving the medication guide image from the pharmacy server. Upon receiving confirmation of reception of the medication guide from the user terminal, the pharmacy server 300 may be notified of medication guide completion.

The storage 180 may store the generated authentication code and store the received medication guide image.

When the inventory management unit 190 inputs at least one of a supply plan, inventory information, and a request quantity, the drug distribution server having received a corresponding order may automatically calculate a consumption quantity and an order quantity based on information input by a pharmacy server, which placed the order, and creates an order sheet and then a corresponding drug may be supplied to a pharmacy that placed the order. The inventory management unit 190 may exchange information with the drug distribution server in real time. When the electronic prescription is received, the inventory management unit 190 may check the inventory of the corresponding drug. When the inventory is insufficient, the inventory management unit 190 may transmit a drug list included in the electronic prescription to the drug distribution server and store information related to drugs included in the drug list. The information related to the drugs may include information on effects and efficacy of a plurality of drugs. In addition, stock quantity information on the drug may be stored, and replenishment scheduling information according to drug replenishment request information of the pharmacy server may be stored. The inventory management unit 190 may request drug replenishment from the drug distribution server when a quantity of drugs stored in a drug packet unit is less than a predetermined quantity. The drug QR code generator 191 is a QR code generated based on a drug list described in the received electronic prescription, and patient information is not included in the generated drug QR code.

The POA generator 192 generates a POA based on the agent code password received from the user terminal 200. The drug cost calculator 195 may calculate a drug cost based on prescription information and request a calculated cost from the cloud server 100. When a delivery QR confirmation is requested from the user terminal, the delivery QR code authentication unit 197 may authenticate a delivery QR code of a courier. When the delivery QR code of the courier is authenticated, the delivery QR code authentication unit 197 may notify the pharmacy server of delivery completion. The communication unit 193 may exchange data with the user terminal, the hospital server, and the pharmacy server through a wired/wireless communication module. The controller 199 controls each configuration of the cloud server.

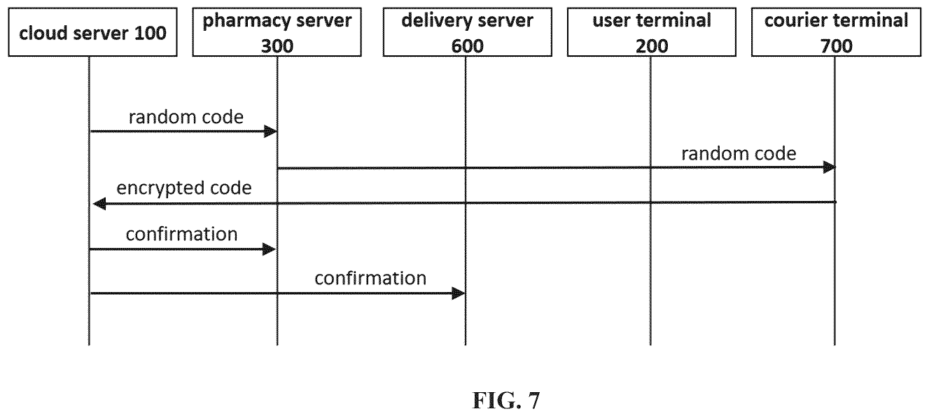

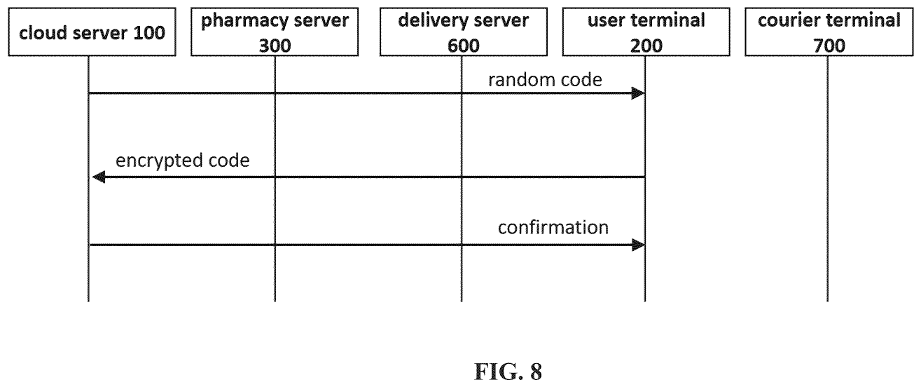

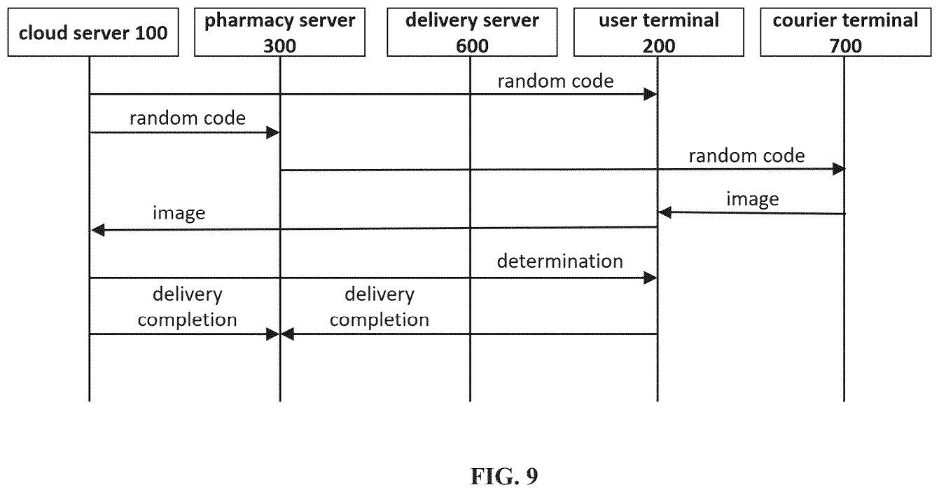

Turning now to FIGS. 7-9, according to another exemplary embodiment, the authentication may be conducted with a random code. In an exemplary embodiment, the cloud server 100 may generate a random code based on a unique ID of the electronic prescription received from the hospital server 200 and transmit the random code to the user terminal 200 and the pharmacy server 300. The cloud server may request identity authentication of a courier terminal 700 from the pharmacy server 300, and then the pharmacy server 300 may transmit the random code to the courier terminal 700. According to an exemplary embodiment, the courier terminal 700 may generate an encrypted code data in response to receipt of the random code and transmit the encrypted code to the cloud server 100. Then, the cloud server 100 may decrypt the encrypted code data to generate a decrypted code data and analyze the decrypted code data and the generated random code to determine if the decrypted code data matches the generated random code data. The cloud server 100 may transmit a confirmation of the identity authentication of the courier terminal 700 to the delivery server 600 and the pharmacy server 300.

According to an exemplary embodiment, the cloud server 100 may further request identity authentication of the user terminal 200. In an exemplary embodiment, the user terminal 200 may generate a encrypted code data in response to receipt of the random code and transmit the encrypted code data to the cloud server 100. Then, the cloud server 100 may decrypt the encrypted code data to generate a decrypted code data, analyzing the decrypted code data and the generated random code to determine if the decrypted code data matches the generated random code data, and the cloud server 100 may transmit a confirmation of the identity authentication of the user terminal to the user terminal 200.

According to an exemplary embodiment, when a user receives the drug from courier, the user terminal 200 may display a message or a functional window indicating arrival in ten minutes. The courier terminal 700 may display an image that is generated, by courier terminal 700 in response to receipt of the random code, including the encrypted code data. Then, the user terminal 200 may read the image from the courier terminal 700. The user terminal 200 may transmit the image to the cloud server 100. Then, the cloud server 100 may decrypt the encrypted data from the image, analyze the decrypted code data from the image and the random code to determine if the decrypted code data from the image matches the generated code data. Then, the cloud server 100 may transmit a determination to the user terminal 200. The user terminal 200 or the cloud server 100 may transmit a delivery completion to pharmacy server 300.

Although the present disclosure has been described with reference to the embodiment shown in the drawings, which is only exemplary, those skilled in the art will understand that various modifications and equivalent other embodiments are possible therefrom. Accordingly, the true technical protection scope of the present disclosure should be determined by the technical spirit of the appended claims

What is claimed is:

1. A method of providing prescription drug preparation, distribution and delivery services through an electronic prescription, the method comprising:
executing, by a user terminal, a patient-only application to select a hospital where to receive medical care and whether to receive remote medical care, and to transmit a result of the selection to a cloud server;
determining, by the cloud server, whether a hospital appointment and remote medical care are possible based on a selection result received from the user terminal, and requesting, by the cloud server, an appointment and remote medical care to a hospital server available of provision of medical care based on a determination result;
proceeding with, by the hospital server, the appointment of the remote medical care and the remote medical care, requesting, by the hospital server, payment of medical expenses from the cloud server, and requesting, by the cloud server, confirmation of medical expenses from the user terminal;
making, by the user terminal, payment for medical care and prescription inquiry, requesting, by the user terminal, drug dispensing from the cloud server, and requesting and receiving, by the cloud server, an electronic prescription from the hospital server;
providing, by the cloud server, a pharmacy list to the user terminal, selecting, by the user terminal, information on a pharmacy, a delivery method, and a delivery time and date, transmitting, by the user terminal, a selection result to the cloud server, transmitting, by the cloud server, the electronic prescription to a pharmacy server, generating, by the cloud server, a random code based on a unique ID of the electronic prescription received from the hospital server, and transmitting, by the cloud server, the random code to the user terminal and the pharmacy server; and
checking, by the pharmacy server, stock based on the received electronic prescription, wherein when a drug included in the electronic prescription is not in stock, the pharmacy server transmits a drug barcode to a drug distribution server, retrieves the drug by checking the drug barcode, and transmits the drug to the pharmacy server, and when the drug included in the electronic prescription is in stock, the pharmacy server calculates a drug cost and requests payment of the drug cost from the user terminal,
the method, further comprising:
in response to proceeding with the payment of the drug cost by the user terminal,
transmitting, by the user terminal, payment completion to the pharmacy server, requesting, by the pharmacy server, a supply from the drug distribution server, and automatically checking, by the drug distribution server, a delay time based on the user's selection by the user terminal;
proceeding with, by the pharmacy server, drug preparation, requesting, by the pharmacy server, checking of delivery information from a delivery server, checking, by the delivery server, drug pickup information, requesting, by the delivery server, confirmation from the pharmacy server, transmitting, by the pharmacy server, the delivery information to the cloud server in response,
requesting, by the cloud server, identity authentication of a courier terminal from the pharmacy server, transmitting, by the pharmacy server, the random code to the courier terminal, generating, by the courier terminal, a first encrypted code data in response to receipt of the random code, transmitting, by the courier terminal, the first encrypted code data to the cloud server, decrypting, by the cloud server, the first encrypted code data to generate a first decrypted code data, and analyzing the first decrypted code data and the generated random code to determine, by the cloud sever, if the first decrypted code data matches the generated random code, and transmitting, by the cloud sever, a first confirmation of the identity authentication of the courier terminal to the delivery server and the pharmacy server, obtaining, by the delivery server, information with respect to receipt of the drug by the courier for delivery, and transmitting, by the delivery server, a delivery notification to the pharmacy server;
transmitting, by the pharmacy server, a prescription drug guide to the cloud server, requesting, by the cloud server, identity authentication of the user terminal, generating, by the user terminal, a second encrypted code data in response to receipt of the random code, transmitting, by the user terminal, the second encrypted code data to the cloud server, decrypting, by the cloud server, the second encrypted code data to generate a second decrypted code data, and analyzing, by the cloud sever, the second decrypted code data and the generated random code to determine if the second decrypted code data matches the generated random code data, and transmitting, by the cloud sever, a second confirmation of the identity authentication of the user terminal to the user terminal,
displaying, by the user terminal, a message indicating arrival in ten minutes, reading, by the user terminal, an image from the courier terminal that is generated, by courier terminal, in response to receipt of the random code, wherein the image includes the first encrypted code data, transmitting, by the user terminal, the image to the cloud server, decrypting, by the cloud server, the first encrypted data from the image, analyzing, by the cloud server, the decrypted code data from the image and the random code to determine if the decrypted code data from the image matches the generated random code, transmitting, by the cloud server, a determination to the user terminal, transmitting, by the user terminal or the cloud server, a delivery completion to pharmacy server,
in response to receiving the delivery completion, checking, by the pharmacy server, a medication guide and requesting, by the pharmacy server, settlement from the delivery server,
wherein the random code of the terminal user is configured to be automatically activated, by the delivery server, ten minutes before the arrival and is deactivated from when the random code is generated until the ten minutes before the arrival.
